(19) United States
(12) Patent Application Publication
(10) Pub. No.: US 2007/0239007 A1
(43) Pub. Date: Oct. 11, 2007

(54) ULTRASOUND METHOD FOR ENHANCED VISUALIZATION OF THERMAL LESIONS AND OTHER FEATURES OF BIOLOGICAL TISSUES

(76) Inventors: Ronald H. Silverman, West Nyack, NY (US); Robert Muratore, Huntington, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/681,242

(22) Filed: Mar. 2, 2007

Related U.S. Application Data
(60) Provisional application No. 60/779,348, filed on Mar. 3, 2006.

(51) Int. Cl. A61B 8/00

(52) U.S. Cl. 600/437; 600/439

(57) ABSTRACT

Systems and methods for visualizing lesions in tissue are provided. The systems and the system and methods exploit the differential scattering of ultrasound waves by normal and lesioned tissues to image the lesions. Spectrum analysis of the harmonics in diagnostic ultrasound waves that are differentially backscattered from lesioned and normal tissues provides image contrast and resolution.

### CROSS-REFERENCE TO RELATED APPLICATIONS

This application claims the benefit of U.S. provisional patent application No. 60/779,348 filed on Mar. 2, 2006, which application is hereby incorporated by reference herein in its entirety.

### STATEMENT REGARDING FEDERALLY SPONSORED RESEARCH OR DEVELOPMENT

The United States government has certain rights in the present invention pursuant to research grant No. RO1 CA84588 awarded by the National Cancer Institute and the National Heart, Lung, and Blood Institute.

### FIELD OF THE INVENTION

The present invention relates to systems and methods of imaging and visualization of biological materials (e.g., medical imaging). In particular, the invention relates to visualization of thermal lesions and other structural features of biological tissues.

### BACKGROUND OF THE INVENTION

High-intensity focused ultrasound (HIFU) technique is a noninvasive extracorporeal technique capable of ablating subsurface structures without injuring intervening tissues. Ultrasonic energy can be applied in a target volume to induce tissue necrosis. The HIFU technique has an advantage over other ablative techniques because the tissue in the acoustic focal volume during HIFU ablation is rapidly damaged by a remote energy source (the ultrasonic transducer), and the intervening tissue is not damaged.

HIFU is being widely investigated for the ablation of diseased tissues with the goal of developing noninvasive treatments of cardiac disease and cancer. Local variations in the acoustic properties of tissue lead to variance in predicted HIFU lesion size and location. However, the HIFU induced lesions can be difficult to visualize ultrasonically. Therefore, clinical applications of HIFU will benefit from an ability to distinguish lesioned tissue from normal tissue, by providing lesion-monitoring information to the surgeon.

Consideration is now directed towards developing imaging and visualization techniques for distinguishing lesioned tissue from normal tissue.

### SUMMARY OF THE INVENTION

Imaging systems and visualization techniques are provided for imaging lesions formed in tissues. The systems and techniques exploit the differential scattering of ultrasound waves by normal and lesioned tissue regions to generate image contrast between the two regions. In particular, the systems and techniques utilize spectrum analysis of diagnostic ultrasound waves that are differentially backscattered from lesioned and normal tissues to image the lesions.

In an exemplary ultrasound imaging system, spectrum analysis of the backscattered signals is performed separately on the frequency ranges centered about the fundamental and the second harmonic or other harmonics. Midband-fit (integrated backscatter) values are converted into pixel values in grayscale images. Thus, three ultrasound images, namely a conventional envelope video image, a Midband fit centered about the fundamental image, and a Midband fit centered about the second harmonic, can be obtained. In studies, the midband fit images of both the fundamental and harmonic offer improved contrast and resolution over conventional imaging.

### BRIEF DESCRIPTION OF THE DRAWINGS

Further objects and advantages of the invention will be apparent from a reading of the following description in conjunction with the accompanying drawings in which:

FIG. 1 is an illustration of a High Intensity Focused Ultrasound (HIFU) transducer device assembly, which can be used to induce lesions in tissue, in accordance with the principles of the present invention. The transducer assembly includes a single element co-axial diagnostic transducer.

FIG. 2 is an illustration of the frequency spectrum of the co-axial diagnostic transducer used in the device assembly of FIG. 1.

FIG. 3 is an illustration of scanning system including the HIFU transducer device assembly with the co-axial diagnostic transducer of FIG. 1, and a three-axis, linear-motion, servomotor controller (xyz DC Servo) arrangement, in accordance with the principles of the present invention.

FIG. 4 is an illustration of an exemplary scanning geometry for visualization of lesions using the scanning system of FIG. 3, in accordance with the principles of the present invention.

FIG. 5 is a block diagram illustrating spectrum analysis of tissue images using the scanning system of FIG. 3, in accordance with the principles of the present invention.

FIG. 6A is an illustration of B-mode, mid band fit (MBF) fundamental and MBF harmonic images obtained by spectrum analysis of images of HIFU-treated tissue, which reveal lesions, in accordance with the principles of the present invention.

FIG. 6B is an illustration of magnified portions the MBF fundamental and harmonic images of FIG. 6A.

FIG. 6C is an illustration of lesion images (MBF fundamental and harmonic images) at different cross sectional planes obtained by virtual slicing, in accordance with the principles of the present invention.

FIG. 7 is an illustration of B-mode, mid band fit (MBF) fundamental and MBF harmonic images taken at two times revealing development of a HIFU-induced popcorn lesion, in accordance with the principles of the present invention. A popcorn lesion is a HIFU induced lesion in which vaporization leads to rapid, often audible, release of a gas body.

FIGS. 8A and 8B are illustration of slope and intercept analysis of the HIFU-induced popcorn lesion of FIG. 7, in accordance with the principles of the present invention.

FIG. 9 is an illustration of another High Intensity Focused Ultrasound (HIFU) transducer device assembly, which can be used to induce lesions in tissue, in accordance with the principles of the present invention. The transducer assembly includes an array co-axial diagnostic transducer, which can be used in a pitch catch configuration.

FIG. 10 is an illustration of the calibration frequency spectrum of the co-axial diagnostic transducer used in the device assembly of FIG. 9.

FIG. 11 is an illustration of scanning system including the HIFU transducer device assembly of FIG. 9, in accordance with the principles of the present invention.

FIG. 12 is an illustration of MBF fundamental and MBF harmonic images obtained by spectrum analysis of pitch catch images, which reveal lesions at a depth in HIFU-treated tissue, in accordance with the principles of the present invention.

### DESCRIPTION OF THE INVENTION

Imaging systems and visualization techniques are provided for distinguishing lesioned from normal tissue. The imaging systems and visualization techniques are based on spectrum analysis of the harmonics in diagnostic ultrasound waves that are differentially backscattered from lesioned and normal tissues. The imaging systems use suitable diagnostic transducers for the receiving backscattered ultrasound waves. The imaging may be conducted in brightness mode (B-Mode), pitch-catch mode, or any other appropriate mode suitable for defining lesion geometry, location and size.

The tissue lesions of interest may be the result of natural processes or may be clinically induced, for example, as part of treatments of cardiac disease and cancer. Such treatments may, for example, use HIFU to ablate subsurface structures without injuring intervening tissues. Ultrasonic energy can be applied in a target volume to induce tissue lesions. The imaging systems and lesion visualization techniques may be used in conjunction with HIFU or other treatment systems by which lesions are induced for treatment.

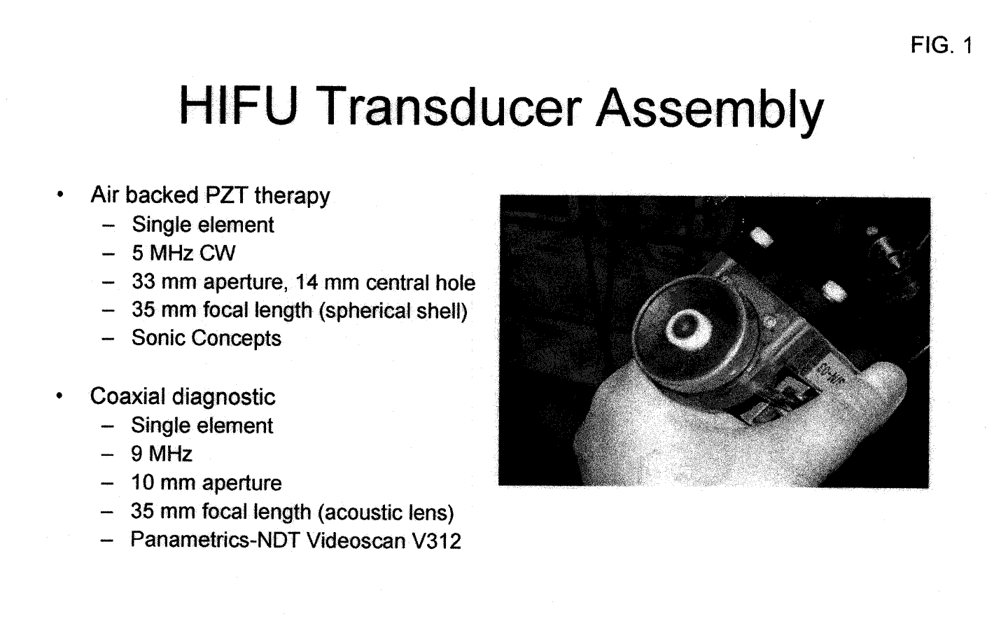

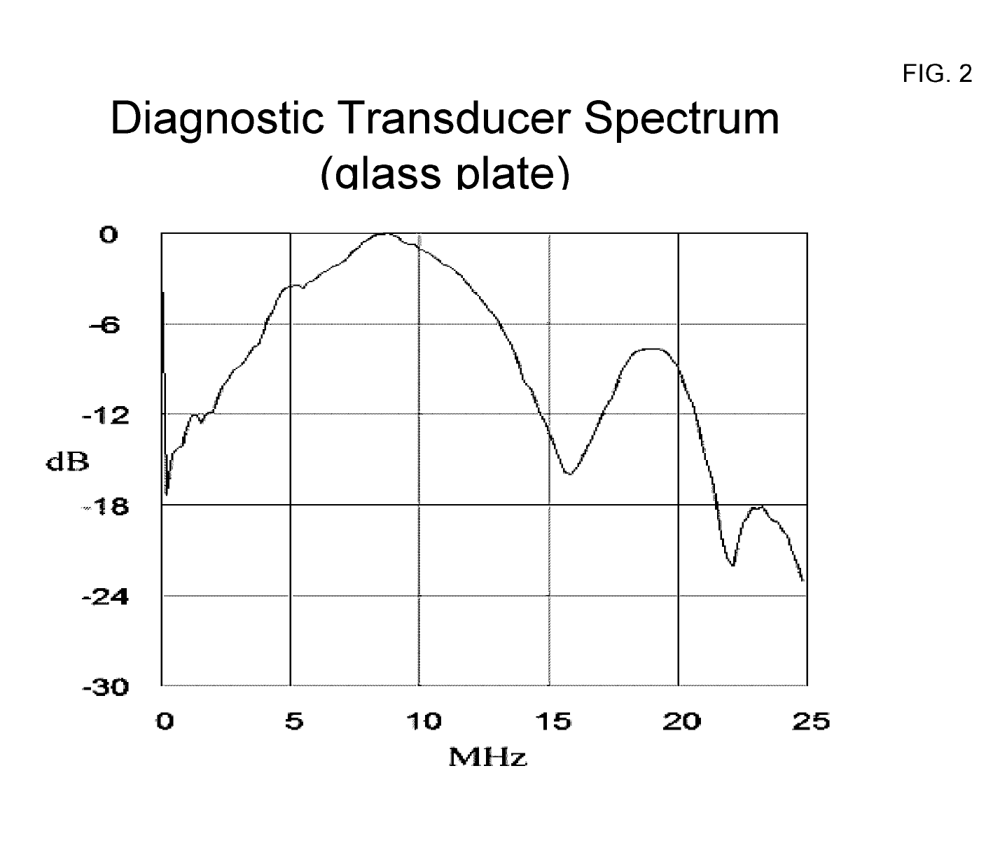

FIG. 1 shows an exemplary combined High Intensity Focused Ultrasound (HIFU) transducer device assembly 100 for inducing lesions and lesion visualization. Assembly 100 includes a High Intensity Focused Ultrasound (HIFU) transducer for therapy and also includes a single element co-axial diagnostic transducer. The HIFU transducer and the diagnostic transducer are suitable commercially available transducers. A suitable HIFU transducer may be an air backed single element PZT transducer (e.g., sold by Sonic Concepts, Bothell, Wash., USA) operating at 5 MHz CW, having a 33 mm aperture and a 35 mm focal length spherical lens. A suitable diagnostic transducer may be a Panametrics-NDT Video Scan V312 transducer operating at 9 MHz and having a 10 mm aperture and a 35 mm focal length acoustic lens. FIG. 2 shows the frequency spectrum of the co-axial diagnostic transducer used in the device assembly of FIG. 1.

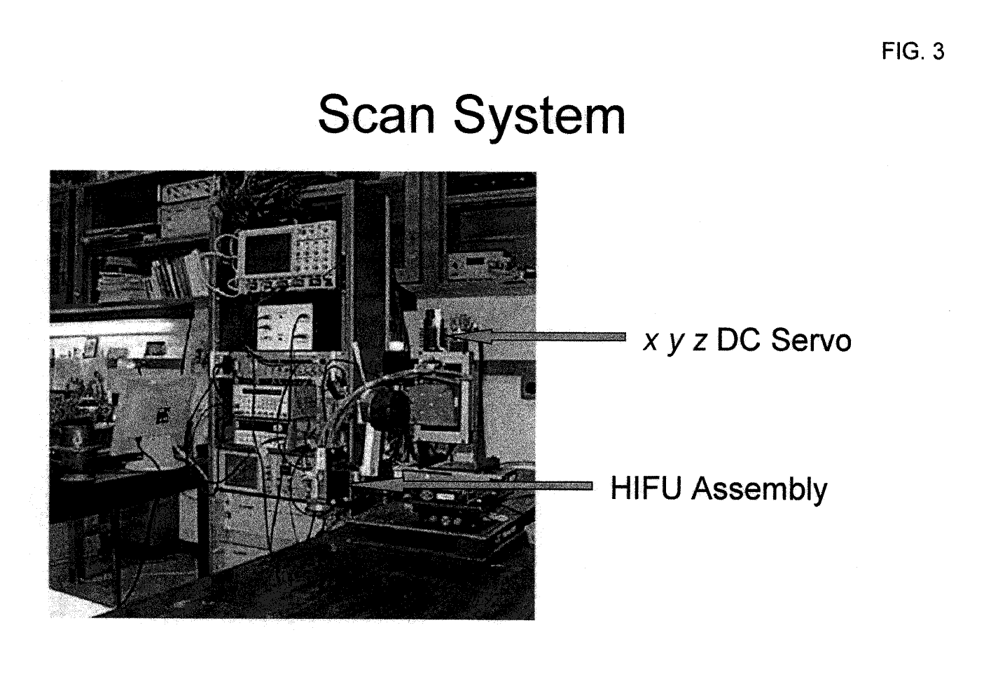

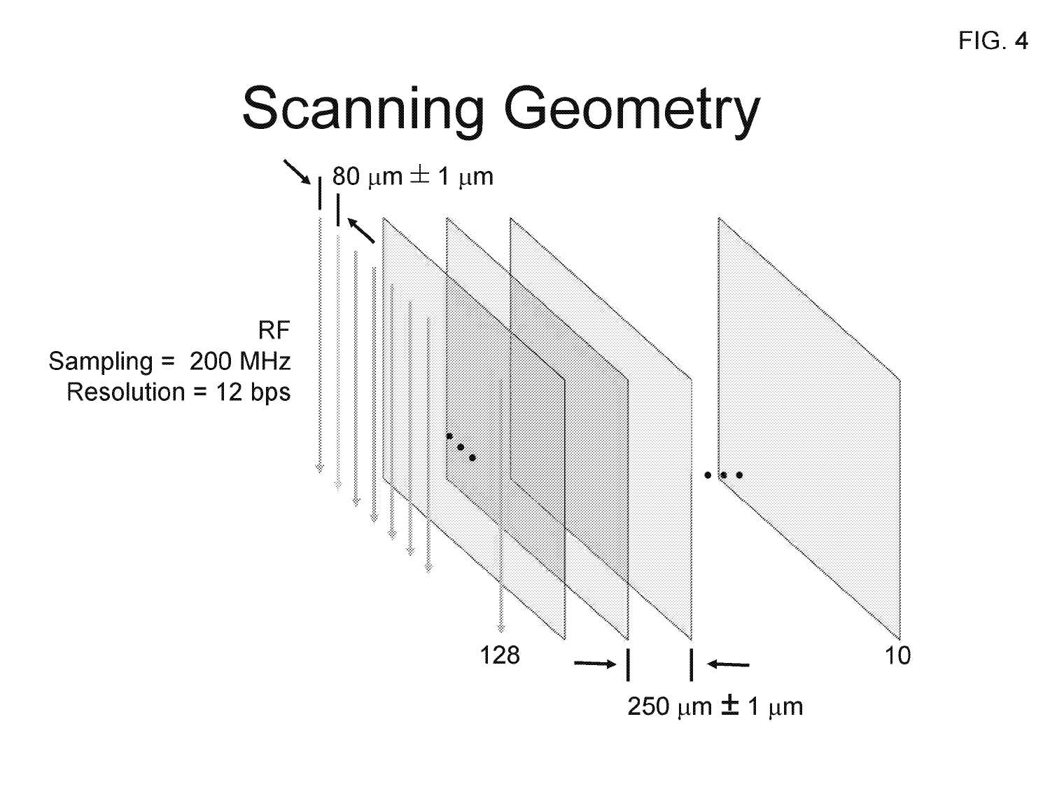

For lesion visualization, assembly 100 is operated in mechanically scanned B-Mode. FIG. 3 shows a scanning system including the HIFU transducer device assembly with the co-axial diagnostic transducer of FIG. 1, and a xyz DC Servo arrangement for stepping the HIFU transducer device assembly in B-scan mode FIG. 4 shows an exemplary scanning geometry for visualization of lesions. The scanning geometry is designed to provide virtual slicing of the specimen in parallel scan planes, each of which can include several suitably spaced apart vectors (FIG. 4).

In a study, lesions were formed by application of high intensity focused ultrasound (HIFU). Lesions were formed using 15-second HIFU exposures (5 MHz CW) at a series of increasing intensities in model tissue (degassed chicken breast in vitro). The tissue was scanned pre- and post-exposure using a confocal 8-MHz diagnostic probe that is incorporated into the HIFU assembly. The diagnostic probe was excited with a monocycle pulse under conditions previously shown to generate a second harmonic comparable in amplitude to the fundamental, and radio frequency (RF) echo-signal data were recorded.

The high intensity focused ultrasound (HIFU) transducer assembly included a PZT spherical shell with a diameter of 35 mm and focal length of 40 mm. A diagnostic transducer with a 9 MHz center frequency was inserted through an aperture in the center of the HIFU assembly providing coaxial diagnostic and HIFU beams. The diagnostic transducer had an aperture of 10 mm and 35 mm focal length.

During operation, a continuous 5 MHz sine wave was amplified by a radiofrequency (RF) power amplifier whose output excited the HIFU transducer. Calibration was performed by measurement of radiation force directed towards an absorbent material using a sensitive balance.

The diagnostic system utilized a custom pulser/receiver (analog bandwidth=75 MHz) that allowed generation of half- or full-cycle (positive or inverted) excitation pulses at frequencies from 5 to 50 MHz. An inverted 9 MHz pulse (40 V) was used to excite the transducer at its fundamental. When reflected from a glass plate in the focal plane, the power spectrum showed a band centered at 9 MHz and a second band at 18 MHz. The 18 MHz band was approximately 8 dB lower in amplitude than the fundamental. When excited at reduced voltage (5 V), the 18 MHz band was reduced to a negligible level. Also, by exciting the transducer at 18 MHz (40 V), an asymmetric pulse spectrum was generated peaking at 18 MHz with progressively lower amplitude at lower frequencies. Hydrophone measurements (Precision Acoustics 0.2 mm aperture needle) were made along the beam axis at 5 mm intervals +/−25 mm relative to the focal plane. Hydrophone results showed that at 40V excitation, the harmonic was at very low levels relative to the fundamental in the near field, reached a maximum at the focal plane and decreased gradually in the far field. At 5 V, the harmonic was generally absent or significantly reduced compared to 40 V. These results are consistent with the 18 MHz peak resulting from non-linear propagation.

Thermal lesions were produced in chicken breast using 15 second exposures of up to 6000 W/cm². In all cases degassed normal saline was used as the coupling medium.

Before and after lesions were generated, the HIFU assembly was scanned across the treatment site and diagnostic data acquired from the coaxial diagnostic ultrasound probe under computer control. The radio frequency backscatter signal was digitized. Radio frequency data was acquired at a sample rate of 200 MHz (12 bits/sample) and stored. Ten parallel scan planes were acquired at intervals of 250 microns. Each plane consisted of 128 vectors (or scan lines) spaced 80 microns apart (FIG. 4).

Spectrum analysis of the RF data was performed separately on the frequency ranges centered about the fundamental and the second harmonic. Midband-fit (integrated backscatter) values were converted into pixel values in grayscale images. Thus, three images were obtained from the RF data:
Envelope video image
Midband fit centered about the fundamental
Midband fit centered about the second harmonic.

In addition, difference images were generated between pre- and post-treatment scan planes for each of the above imaging modalities. For each modality, statistical contrast was determined between the lesion site and adjacent untreated tissues. These findings indicate that midband fit images of both the fundamental and harmonic offer improved contrast versus conventional imaging.

Post-processing involved generation of a number of image types:
Conventional gray-scale signal envelope;
Spectral parameter images (midband fit (MBF), spectral slope and intercept images) restricted to −12 dB fundamental band (3-15 MHz); and
Spectral parameter images (midband fit, spectral slope and intercept images) restricted to −12 dB second harmonic band (16-23 MHz)

Spectral analysis was performed using a sliding window 64 samples in length by 3 vectors in width. The analysis window was rastered over the image and spectra determined in overlapping regions of interest spaced at 4 sample intervals along each vector. Spectra were determined as follows:
RF data multiplied by Hamming function
Perform fast Fourier transform (FFT)
Divide FFT by glass plate calibration spectrum FFT (deconvolve)
Multiply by appropriate filter
Determine calibrated power spectrum in appropriate frequency band
Determine linear best fit equation to spectrum
Determine slope, intercept and amplitude at midband frequency of calibrated power spectrum
Generate pixel values for parameters using color scales for slope and intercept and gray scale for MBF. (Color or gray scale consists of 256 levels thresholded just above noise level and encompassing full range of intensity values.)
Generate 512×512 pixel output slope, intercept and MBF parameter images.

HIFU lesions were poorly visualized in conventional images generated from the envelope of the echo data. MBF parameter images centered at the fundamental were somewhat better than the conventional envelope, but MBF images centered at the harmonic showed best contrast and delineation of lesion boundaries. MBF images formed from an emitted 18 MHz pulse were not as effective in lesion visualization as harmonic mode.

MBF harmonic images can significantly improve contrast and resolution compared to conventional envelope images or images generated at the fundamental.

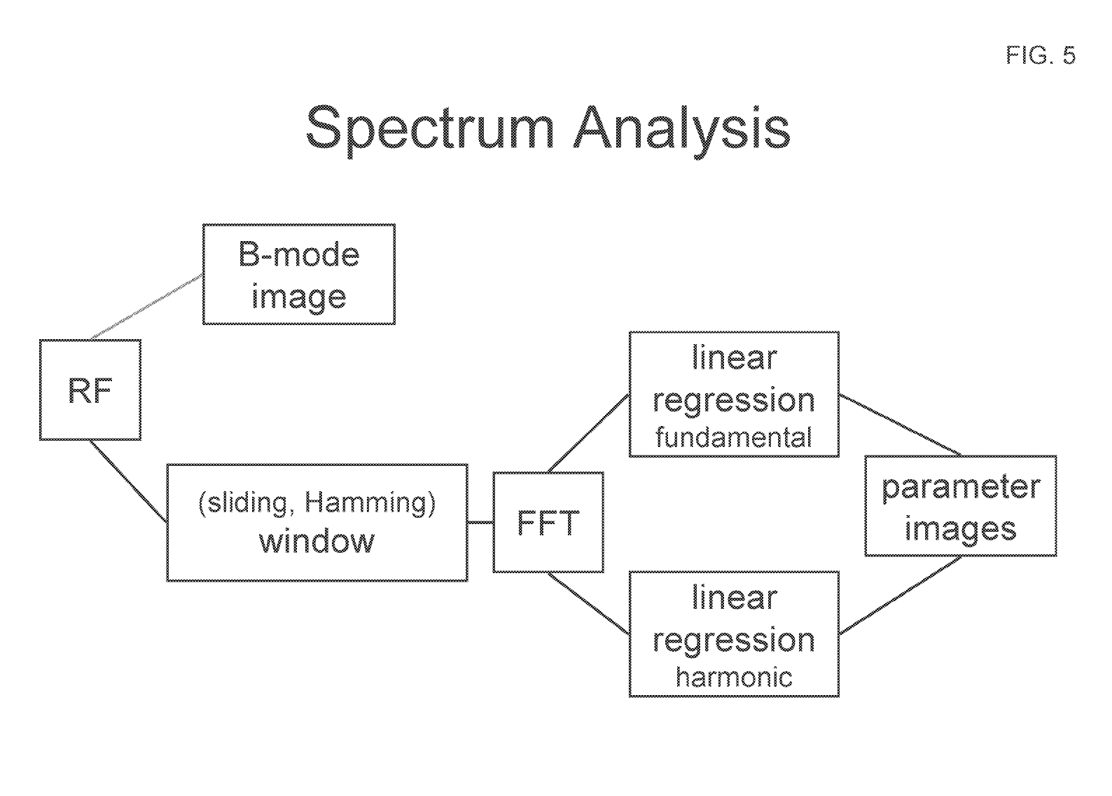

FIG. 5 summarizes processing blocks or functions in the spectrum analysis of the ultrasound images. Block 510 represents the brightness mode(B-mode) two-dimensional ultrasound image. Block 520 represents acquisition of the radio frequency backscatter signal data during imaging. Next, the frequency power spectrum of the backscattered RF-signals is calculated. Each scanline is smoothed (e.g., by applying a Hamming window, Block 530) and the power spectrum calculated by applying Fast Fourier Transform (FFT) (Block 540). The FFT power spectrum is divided in N frequency bands corresponding to the fundamental frequency and N−1 harmonic frequencies, respectively (e.g., N=2, fundamental frequency band 4-15 MHz and second harmonic band 16-22 MHz bands). The spectra in each of the two bands are fitted at mid band by linear regression (Block 550) and normalized to generate parametric images (Block 560).

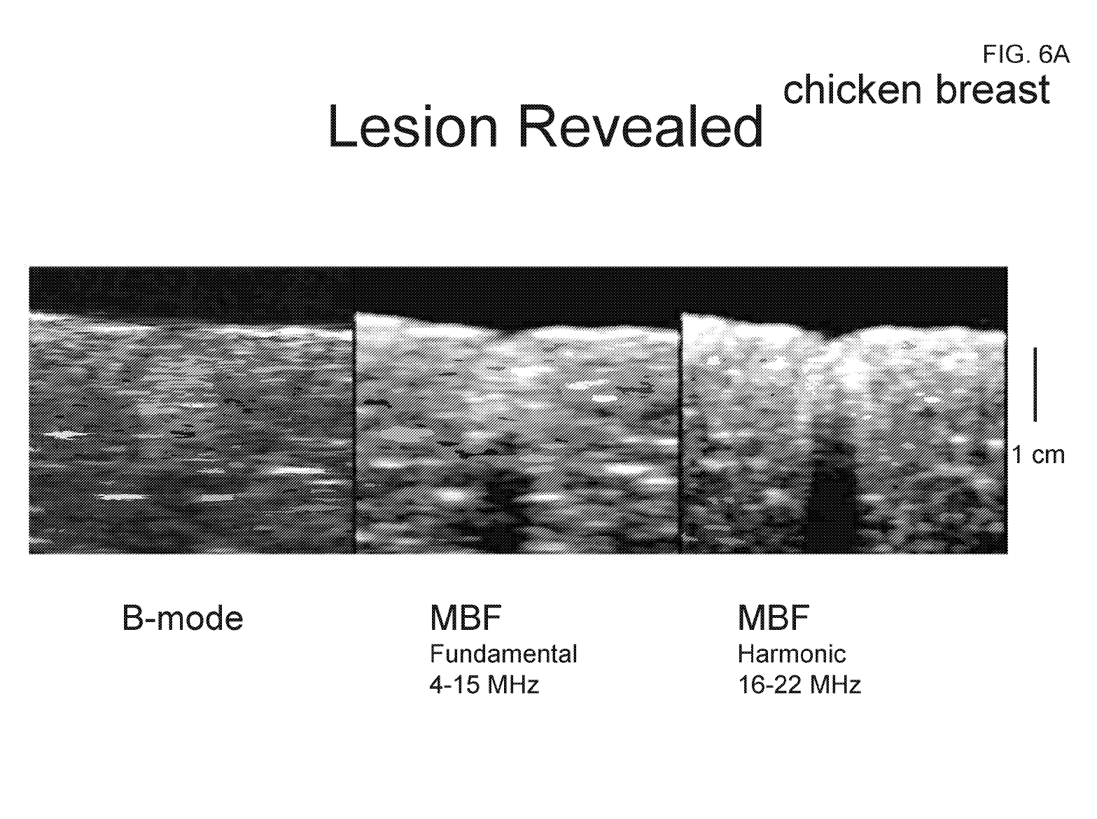

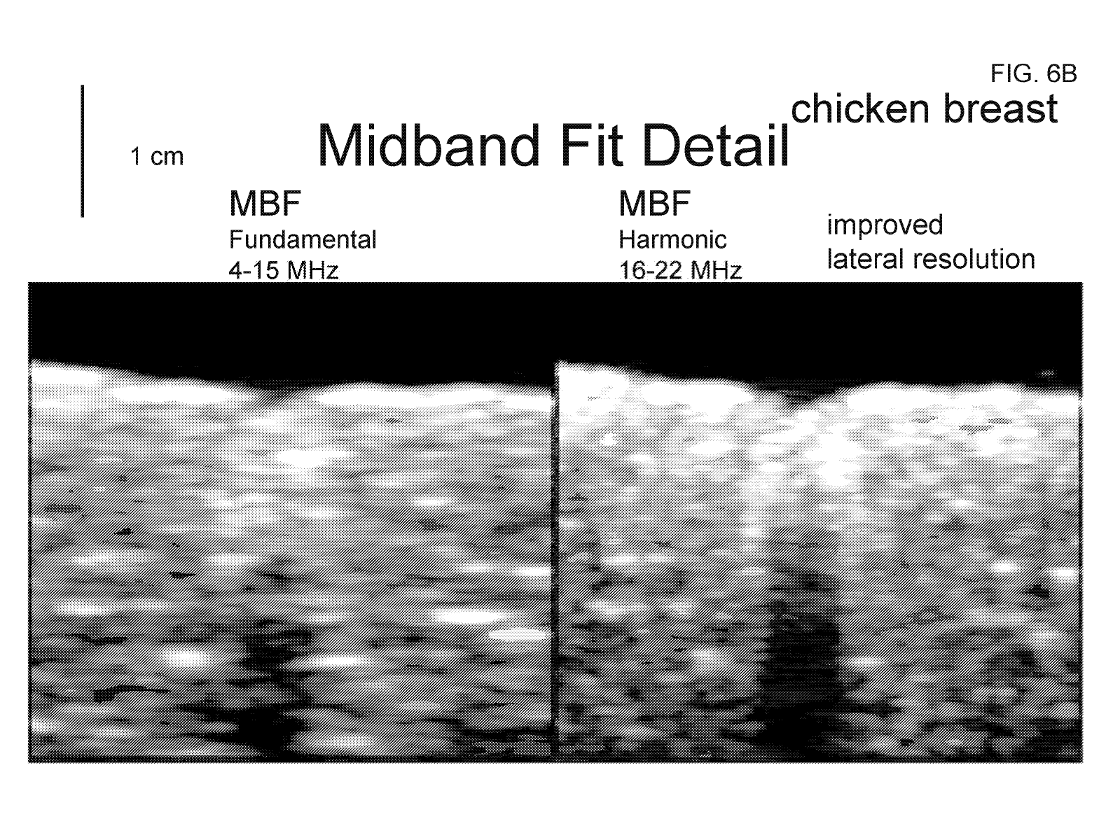

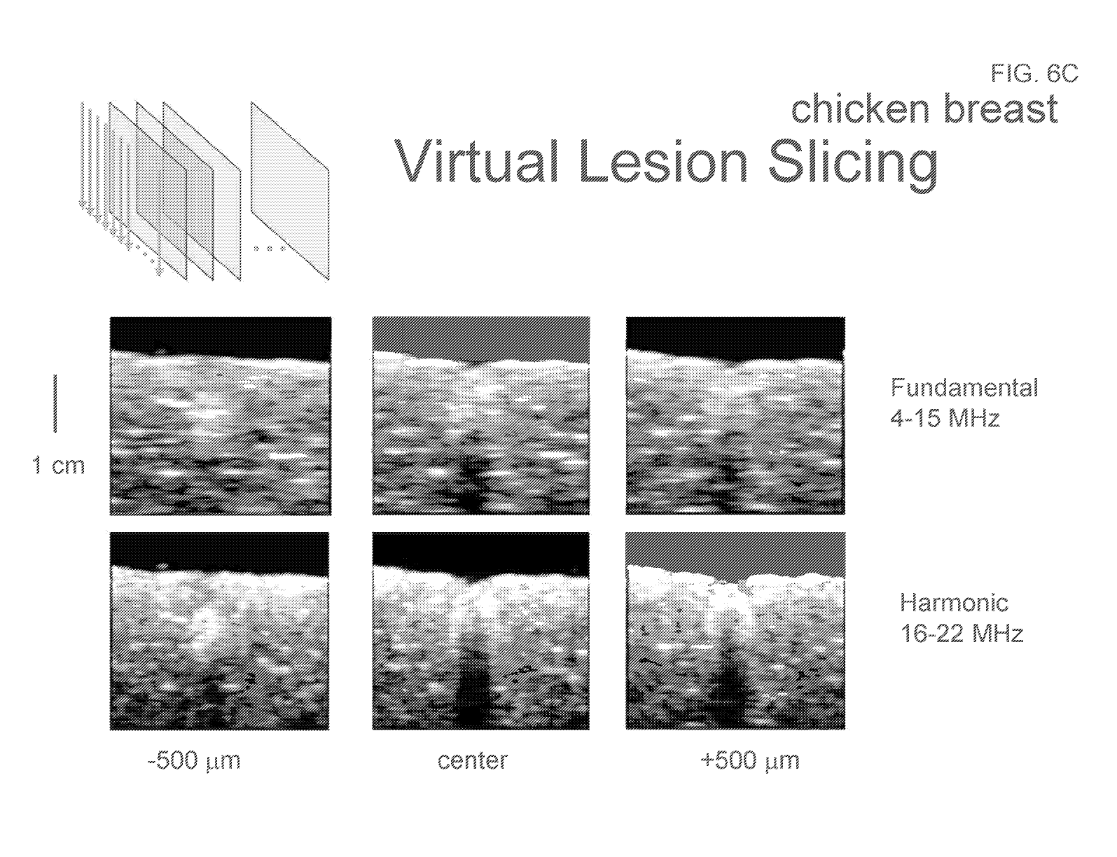

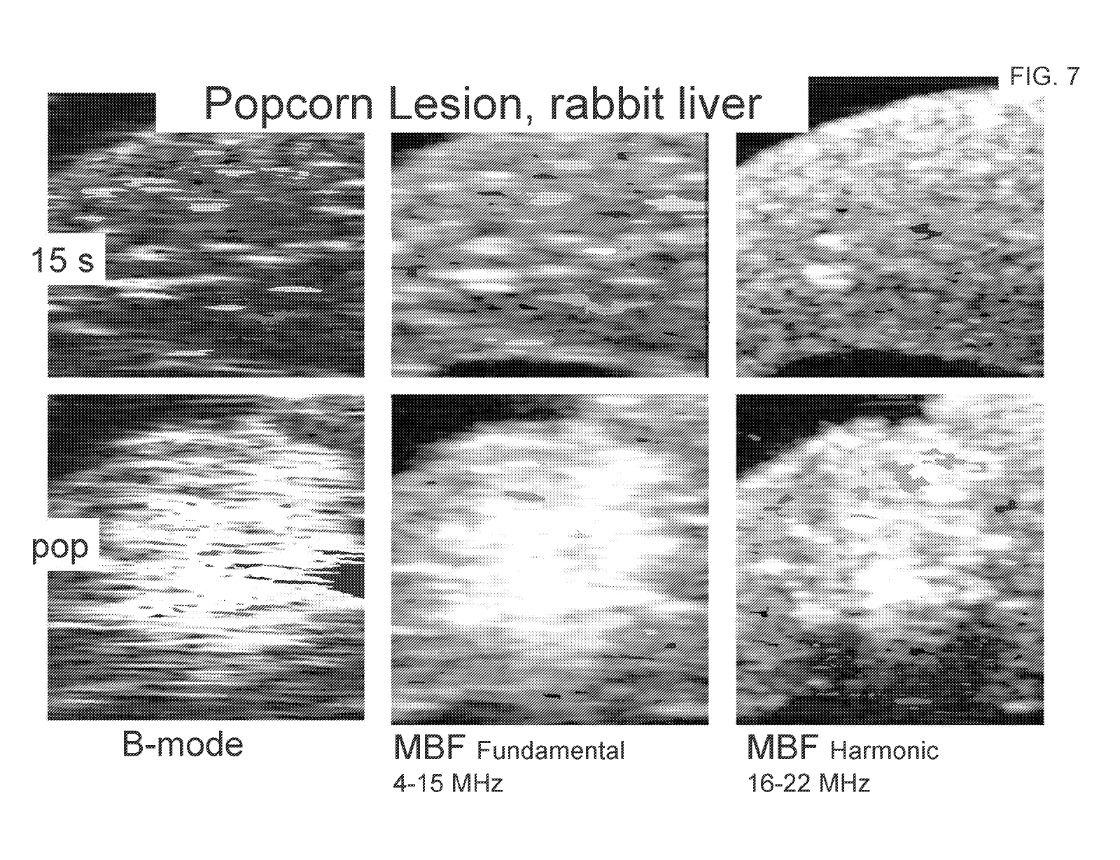

FIG. 6A shows B-mode, mid band fit (MBF) fundamental and MBF harmonic images obtained by spectrum analysis of images of HIFU-treated chick breast tissue in the study. FIG. 6B shows magnified portions the same MBF fundamental and harmonic images. Comparison of the images indicates the presence of lesions in the HIFU-treated chick breast tissue.

FIG. 6C shows MBF fundamental and harmonic images of the lesion in different cross-sectional planes obtained by virtual slicing. Comparison of the images obtained in different cross sectional planes allows determination of the size (e.g., lateral size) of the lesion.

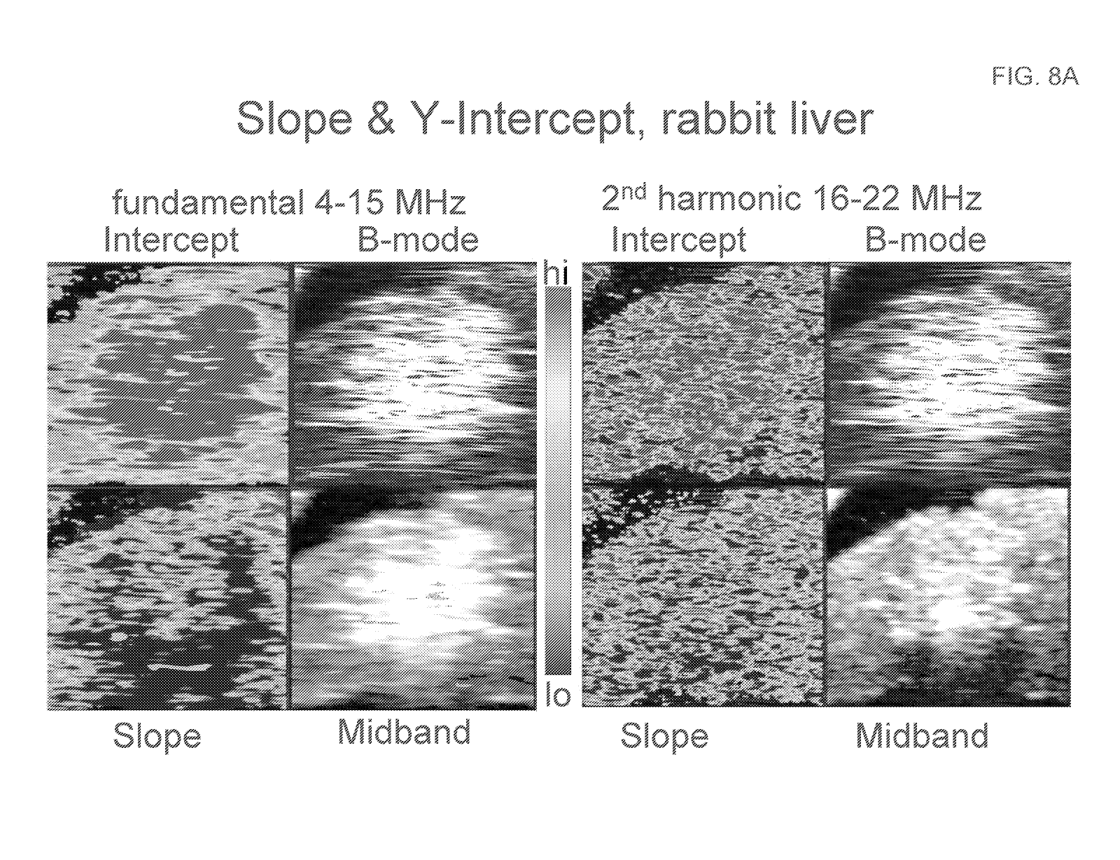

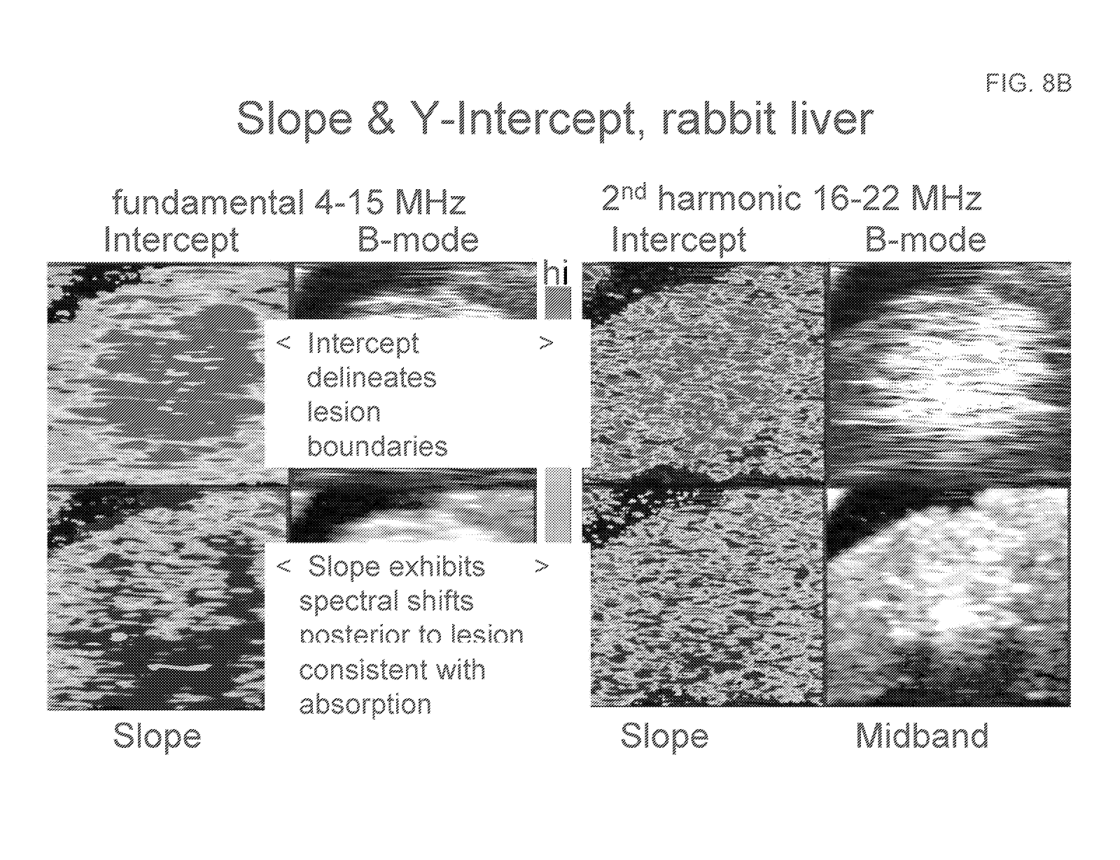

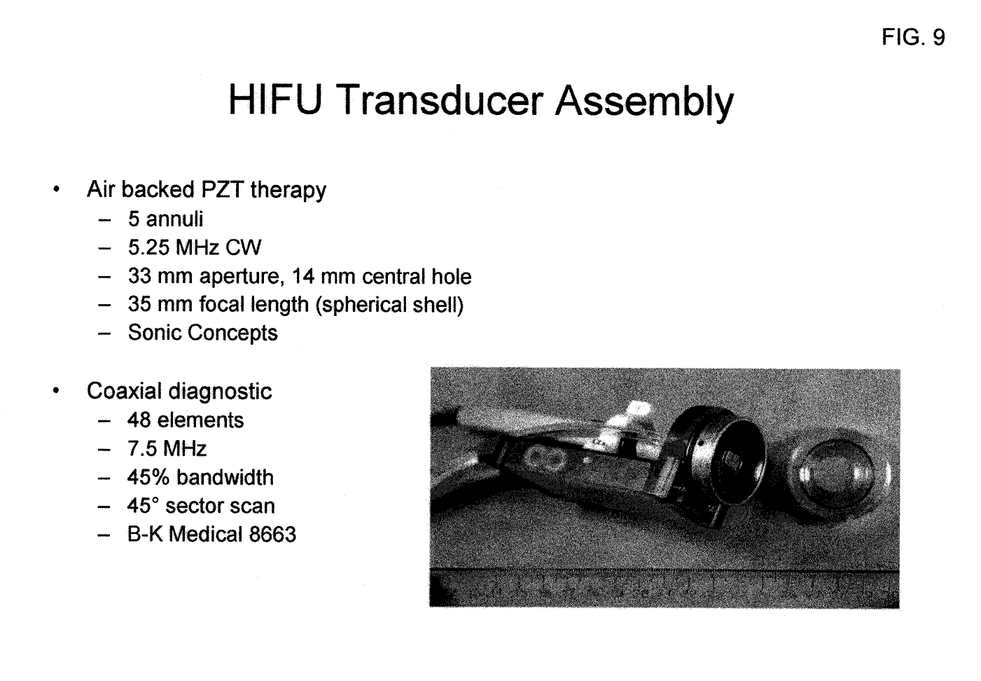

FIG. 8 shows B-mode, MBF fundamental and MBF harmonic images, which reveal the development of an HIFU induced popcorn lesion in a rabbit tissue specimen. The images are shown in two rows at a lower and a higher magnification, respectively. The lateral size of the lesion can be determined by slope and intercept analysis of the image intensity. FIGS. 9A and 9B show the slope and Y-intercept images corresponding to the MBF fundamental and MBF harmonic images of the HIFU-induced popcorn lesion (FIG. 8). The intercept image delineates the lesion boundaries. The slope image exhibits spectral shifts posterior to the popcorn lesion that are consistent with absorption.

The inventive visualization systems and techniques can be used in a pitch-catch configuration that may allow determination of the location, size and orientation of a lesion without scanning or relocating the transducer.

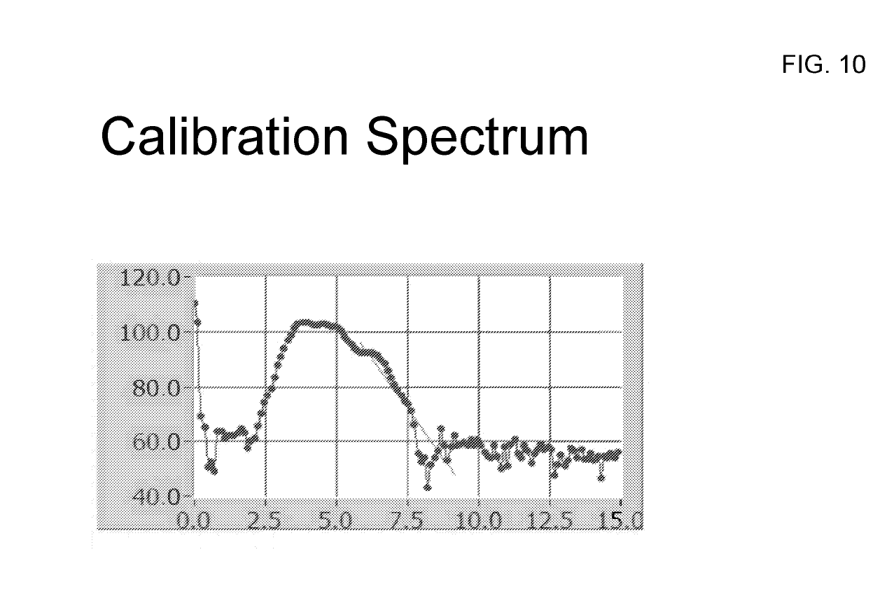

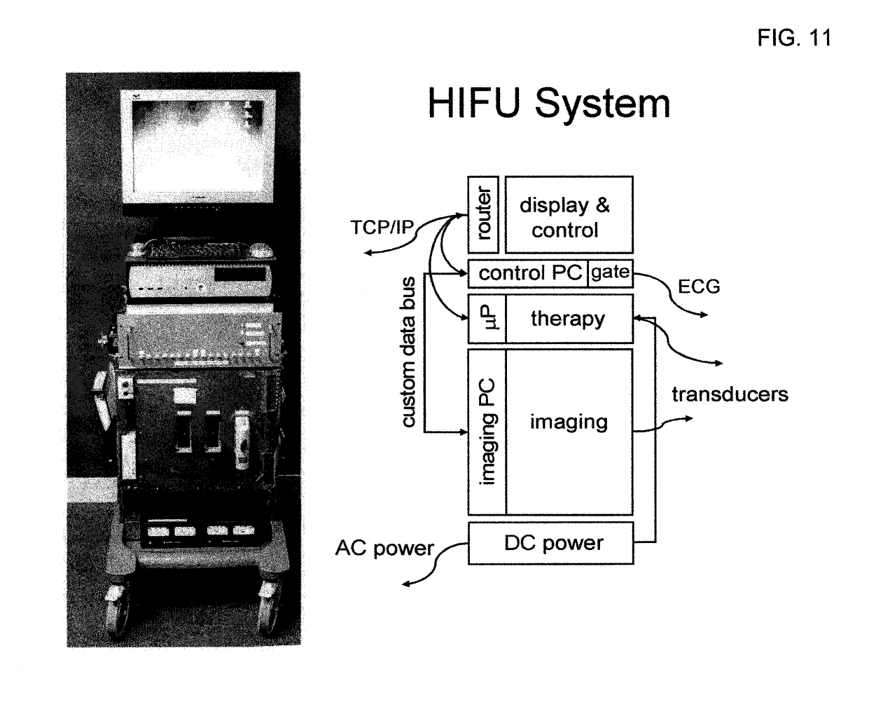

FIG. 10 shows an exemplary combined High Intensity Focused Ultrasound (HIFU) transducer device assembly 1000 for inducing lesions and lesion visualization in a pitch-catch mode. Assembly 1000, like assembly 100, includes a High Intensity Focused Ultrasound (HIFU) transducer for therapy but includes a multi element or array co-axial diagnostic transducer. The HIFU transducer and the diagnostic transducer are suitable commercially available transducers. A suitable HIFU transducer may be an air backed single element PZT transducer (e.g., sold by Sonic Concepts, Bothell, Wash., USA) operating at 5 MHz CW, having a 33 mm aperture and a 35 mm focal length spherical lens. A suitable diagnostic transducer may be a B-K Medical 8663 transducer with 48 array elements operating at 7.5 MHz with a 45 deg. Sector scan. FIG. 11 shows the calibration frequency spectrum of the co-axial diagnostic transducer used in the device assembly of FIG. 10. For lesion visualization, assembly 1000 is operated in pitch-catch mode.

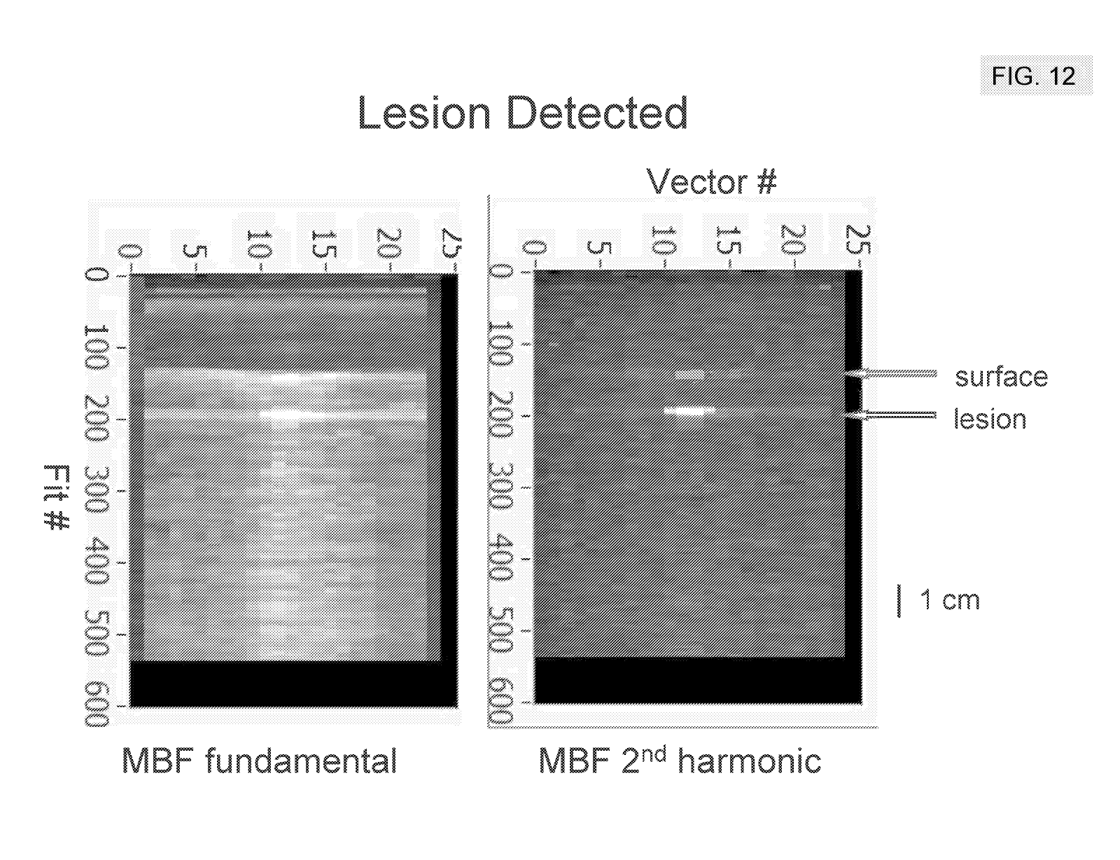

FIG. 11 shows a scanning system including the HIFU transducer device assembly 1000 with the multi-element co-axial diagnostic transducer of FIG. 10. FIG. 12 is a block diagram illustrating spectrum analysis of tissue images using the scanning system of FIG. 11. The spectrum analysis may be conducted in a manner similar to that shown in FIG. 5, with the obvious adjustment of the fundamental frequency band and the harmonic frequency bands to 3-7.5 MHz and 7.5-12-5 MHz, respectively, corresponding to nominal 7.5 MHz operation of the diagnostic transducer.

In a study, lesions were formed in calf heart tissue specimens by application of high intensity focused ultrasound (HIFU). The calf heart tissue specimens were prepared by cutting commercial calf heart left ventricle into strips, which were immerse din PBS at 24 deg. C. Lesions were formed in a calf tissue specimen using HIFU (5.25 MHz CW, 58 W_{acoustic} corresponding to 43 kW/cm² nominal at SPTA_{-3db}) focused at 1 cm below the epicardial surface. 10 pulses of HIFU each 200 ms long were used. The tissue specimen was scanned pre- and post-exposure using the 7.5 MHz diagnostic transducer in pitch-catch mode. The specimen was irradiated with high intensity, short duration therapy pulses each about 400 microseconds long. The RF-backscatter signals were received with the diagnostic transducer and digitized at 30 MHZ.

For example, other possible embodiments of the inventive systems and techniques may use hardware implementations of the method such as floating point gate arrays to provide real-time presentation of the modified data to the operator. Other possible embodiments may use data obtained through detection of transmitted ultrasound signals rather than backscattered ultrasound signals.

1. A method for preparing ultrasonic imaging of tissue, the method comprising:
using an ultrasound generator to generate ultrasound waves having a nominal frequency;
irradiating the tissue with the ultrasound waves having the nominal frequency; and
recording and digitizing image data comprising RF signals that are backscattered from the tissue;
conducting spectrum analysis of the backscattered RF signals to separate image data corresponding to at least one of N harmonic frequency bands (N=0, N=N−1); and
displaying an image based on the separated data corresponding to the at least one of N harmonic frequency bands, whereby lesions can be distinguished from normal tissue.

2. The method of claim 1 wherein irradiating the tissue with the ultrasound waves having the nominal frequency comprises scanning the tissue in brightness mode (B-Mode).

3. The method of claim 1 wherein irradiating the tissue with the ultrasound waves having the nominal frequency comprises scanning the tissue in pitch-catch mode.

4. The method of claim 1 wherein displaying an image based on the separated data, comprises converting image data into pixel values in grayscale for display.

5. The method of claim 4 wherein converting image data into pixel values in grayscale for display comprises converting midband fit data into pixel values in grayscale for display.

6. The method of claim 1 wherein the at least two of the harmonic frequency bands correspond to a fundamental frequency and a second harmonic frequency.

7. The method of claim 1 further comprising using backscattered RF signal envelop data to form an envelope video image.

8. The method of claim 1 further comprising generating images before and after a treatment of the tissue, and generating pre- and post-treatment difference images.

9. The method of claim 1 further comprising generating two images corresponding to any two of an envelope video image and the N harmonic frequency bands; and comparing the two images so that lesions can be distinguished from normal tissue.

10. The method of claim 1 further comprising determining the lateral extent of a lesion by slope and intercept analysis of image intensity.

11. The method of claim 1 further comprising determining the depth of a lesion by determining attenuation of ultrasound caused by overlying tissue in a pitch and cath mode.

12. A system for imaging lesions in tissue, the system comprising:
an ultrasound transmitter for irradiating the tissue at a nominal frequency;
an ultrasound receiver for receiving RF signals backscattered from the tissue;
a power spectrum analyzer configured to process received backscattered RF signals to separate image data corresponding to at least one of N harmonic frequency bands (N=0, . . . , N=N−1), and
to display an image based on the separated data corresponding to the at least one of N harmonic frequency bands, whereby lesions can be distinguished from normal tissue due to the differential scattering of ultrasound by lesioned and normal tissue.

13. The system of claim 12 wherein the ultrasound transmitter and receiver comprise an ultrasound transducer configured to irradiate the tissue with the ultrasound waves in brightness mode (B-Mode).

14. The system of claim 12 wherein the ultrasound transmitter and receiver comprise a multi-element ultrasound transducer configured to irradiate the tissue with the ultrasound waves in pitch-catch mode.

15. The system of claim 12 wherein the power spectrum analyzer further comprises algorithms for converting image data into pixel values in grayscale for display.

16. The system of claim 12 wherein the power spectrum analyzer further comprises algorithms for converting midband fit data into pixel values in grayscale for display.

17. The system of claim 12 wherein the at least two of the harmonic frequency bands correspond to a fundamental frequency band and a second harmonic frequency.

18. The system of claim 12 further comprising algorithms for converting backscattered RF signal envelop data into envelop video image data.

19. The system of claim 12 further comprising algorithms for generating two images corresponding to any two of an envelope video image and the N harmonic frequency bands; and comparing the two images so that lesions can be distinguished from normal tissue.

20. The system of claim 12 further comprising algorithms for generating images before and after a treatment of the tissue, and generating pre- and post-treatment difference images.

21. The system of claim 12 further comprising algorithms for determining the lateral extent of a lesion by slope and intercept analysis of image intensity.

22. The system of claim 12 further comprising algorithms for determining the depth of a lesion by determining attenuation of ultrasound caused by overlying tissue in a pitch and catch model.
